(19) United States
(12) Patent Application Publication
(10) Pub. No.: US 2019/0148025 A1
(43) Pub. Date: May 16, 2019

(54) PATIENT THERAPY MANAGEMENT AND COACHING SYSTEM

(71) Applicant: MEDTRONIC MINIMED, INC., Northridge, CA (US)

(72) Inventors: Michael P. Stone, Long Beach, CA (US); Kevin E. Velado, Northridge, CA (US); Siddharth Arunachalam, Northridge, CA (US); Boyi Jiang, Northridge, CA (US); Jacob Bowland, Pasadena, CA (US); Nolan Lindeke, Northridge, CA (US); Pratik Agrawal, Porter Ranch, CA (US); Raymond C. Liu, Northridge, CA (US); Kristin S. Sharma, Mount Pleasant, MI (US); Yuxiang Zhong, Arcadia, CA (US); Jinghua Chen, Encino, CA (US); Ana Romero, Palmdale, CA (US)

(21) Appl. No.: 16/189,902

(22) Filed: Nov. 13, 2018

Related U.S. Application Data
(60) Provisional application No. 62/586,672, filed on Nov. 15, 2017.

(51) Int. Cl. G16H 80/00; A61B 5/00; A61B 5/145; A61M 5/00; G16H 20/17; G16H 50/20

(52) U.S. Cl. CPC G16H 80/00; A61B 5/0022; A61B 5/743; A61B 5/14532; G16H 50/30; G16H 20/17; G16H 50/20; A61B 5/0004; A61M 2205/3584; A61M 5/003

(57) ABSTRACT

The system disclosed here includes a computer-implemented patient therapy management and coaching system, a database system to collect and maintain patient data associated with a plurality of medical device users that includes a trainee patient assigned to a coach, and a user device communicatively coupled to the patient therapy management and coaching system and associated with the coach. The management and coaching system is operative to: receive a patient request for coaching; process the request to automatically identify goals to be achieved by the trainee patient; communicate the goals to the coach's user device; receive an accepted goal selected from the identified goals; create a patient coaching program for the accepted goal; generate insight messages based on patient data collected for the trainee patient, the generated insight messages related to the patient coaching program; and deliver the generated insight messages to the user device associated with the coach.

### CROSS-REFERENCE TO RELATED APPLICATION

This application claims the benefit of U.S. provisional patent application Ser. No. 62/586,672, filed Nov. 15, 2017. The content of the referenced application is incorporated by reference herein.

### TECHNICAL FIELD

Embodiments of the subject matter described herein relate generally to systems and methods for diabetes therapy management. More particularly, embodiments of the described subject matter relate to a computer-based patient coaching system and related operating methodology that help patients meet specified behavioral goals and/or medical outcome goals.

### BACKGROUND

Portable medical devices are useful for patients that have conditions that must be monitored on a continuous or frequent basis. For example, diabetics are usually required to modify and monitor their daily lifestyle to keep their blood glucose (BG) in balance. Individuals with Type 1 diabetes and some individuals with Type 2 diabetes use insulin to control their BG levels. To do so, diabetics are advised to routinely keep strict schedules, including ingesting timely nutritious meals, partaking in exercise, monitoring BG levels daily, and adjusting and administering insulin dosages accordingly.

The prior art includes a number of fluid infusion devices and insulin pump systems that are designed to deliver accurate and measured doses of insulin via infusion sets (an infusion set delivers the insulin through a small diameter tube that terminates at, e.g., a cannula inserted under the patient's skin). In lieu of a syringe, the patient can simply activate the insulin pump to administer an insulin bolus as needed, for example, in response to the patient's high BG level. A patient can monitor BG levels using a BG meter or measurement device and by using a continuous glucose sensor if so desired.

In practice, many processes and behaviors result in fluctuations in BG levels. Commonly recognized processes and factors impacting BG levels include food, exercise, disease (acute or chronic), medication (insulin, oral, etc.), and stress and sleep patterns, among others. Furthermore, behavioral and environmental factors such as the time of the day, attentiveness to therapy, and insulin pump maintenance, can provide additional quantitative indications of the underlying factors impacting glucose control. Current reporting tools available to diabetes patients and their caregivers collect and analyze data in a way that is intended to address an individual patient's particular glycemic outcome.

Accordingly, it is desirable to have a system and related methodologies that provide patient coaching and assistance, particularly with respect to diabetes patients using insulin infusion systems. Furthermore, other desirable features and characteristics will become apparent from the subsequent detailed description and the appended claims, taken in conjunction with the accompanying drawings and the foregoing technical field and background.

### BRIEF SUMMARY

A method of creating and managing interactive patient coaching programs is disclosed. An embodiment of the method involves: collecting patient data associated with a plurality of medical device users, wherein the plurality of medical device users includes a trainee patient assigned to a coach; receiving a patient request for coaching; processing the request with a computer-based system to automatically identify goals to be achieved by the trainee patient; communicating the identified goals to a computer-based user device associated with the coach; receiving, at the computer-based system, an accepted goal selected from the identified goals; creating, with the computer-based system, a patient coaching program for the accepted goal; generating insight messages based on patient data collected for the trainee patient, the generated insight messages related to the patient coaching program; and delivering the generated insight messages to the computer-based user device associated with the coach.

Also disclosed is a computer-implemented patient therapy management and coaching system. An embodiment of the system includes: at least one processor device; and a non-transitory processor-readable medium operatively associated with the at least one processor device. The processor-readable medium has executable instructions configurable to cause the at least one processor device to perform a method that involves: collecting patient data associated with a plurality of medical device users, wherein the plurality of medical device users includes a trainee patient assigned to a coach; receiving a patient request for coaching; processing the request to automatically identify goals to be achieved by the trainee patient; communicating the identified goals to a computer-based user device associated with the coach; receiving an accepted goal selected from the identified goals; creating a patient coaching program for the accepted goal; generating insight messages based on patient data collected for the trainee patient, the generated insight messages related to the patient coaching program; and delivering the generated insight messages to the computer-based user device associated with the coach.

Another system is also disclosed herein. An embodiment of the system includes: a computer-implemented patient therapy management and coaching system; a database system, associated with the patient therapy management and coaching system, to collect and maintain patient data associated with a plurality of medical device users, wherein the plurality of medical device users includes a trainee patient assigned to a coach; and a computer-implemented user device communicatively coupled to the patient therapy management and coaching system, the user device associated with the coach. The patient therapy management and coaching system is operative to: receive a patient request for coaching; process the request to automatically identify goals to be achieved by the trainee patient; communicate the identified goals to the user device associated with the coach; receive an accepted goal selected from the identified goals; create a patient coaching program for the accepted goal; generate insight messages based on patient data collected for the trainee patient, the generated insight messages related to the patient coaching program; and deliver the generated insight messages to the user device associated with the coach.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a simplified block diagram representation of an operating environment than includes a patient therapy management and coaching system;

FIG. 2 is a simplified block diagram representation of an exemplary embodiment of a computer-based or processor-based device suitable for deployment in the operating environment shown in FIG. 1;

FIG. 3 is a diagram that illustrates a typical use case that can be supported by the patient therapy management and coaching system shown in FIG. 1;

FIG. 4 is a flow chart that illustrates an exemplary embodiment of a methodology for creating and managing interactive patient coaching programs; and

FIG. 5 is a diagram of a patient timeline.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or instructions that perform the various tasks. In certain embodiments, the program or code segments are stored in a tangible processor-readable medium, which may include any medium that can store or transfer information. Examples of a non-transitory and processor-readable medium include an electronic circuit, a semiconductor memory device, a ROM, a flash memory, an erasable ROM (EROM), a floppy diskette, a CD-ROM, an optical disk, a hard disk, or the like.

The following description relates to a computer-implemented diabetes patient support and coaching system that uses real-time intelligent data processing algorithms that enable proactive support to improve patient outcomes and increase user engagement, while reducing the cost of operation (by using analyzed patient data to prioritize patient outreach and to provide effective patient training with respect to medical device operation and maintenance). Although not limited to any particular use case, the end users for the exemplary embodiment described here are healthcare professionals, caregivers, doctors, medical device manufacturer/company representatives, clinicians, patient coaches, etc. That said, the system described here can be utilized to provide some or all of the coaching-related content to patients if so desired.

The exemplary embodiment disclosed herein is a cloud-based architecture in that most of the processor-intensive tasks are performed by one or more server systems that communicate with remotely located computer-based user devices. In practice, the disclosed system can obtain and process patient data for a population of different patients under the care of different physicians. The following description, however, focuses on a typical use case where the coaching platform is being used to assist one particular patient. Patient data can originate from various sources, including insulin infusion devices, continuous glucose sensor devices, mobile client devices, patient owned or operated computer systems, activity tracker devices, navigation or global positioning system (GPS) devices, etc. Patient data is processed and analyzed to generate therapy-related content that can be displayed on a website and/or via a web-based application.

An exemplary embodiment of the system described here can utilize an online (web browser based) application that serves as a patient management and coaching tool. To this end, the system can be implemented with a website that provides diabetes therapy recommendations, advice, patient status or progress reports, and/or guidance to the patient's coach (e.g., a medical device company representative or support personnel, a caregiver, a healthcare professional, a parent, etc.) for purposes of driving better glycemic outcomes, patient behavior, and proper medical device usage. In accordance with certain implementations, the web-based tool provides coaches with valuable information (recommendations) for patients on insulin therapy, and enables coaches to easily track patient progress in meeting certain behavioral and/or glycemic outcome goals. In practice, at least some of the content generated and provided by the system is based on the analysis of patient data collected for at least the patient under consideration. Actionable recommendations lead to better patient engagement and improve care efficiency. The web-based coaching tool is an ideal platform to leverage machine learning algorithms that analyze patient data to help improve patient behavior and glycemic outcomes.

The web-based tool presents the end user with a variety of interactive web pages that include graphical elements, menus, text entry fields, search fields, output/results screens, and the like. For example, the web-based tool may include, without limitation: output pages or displays that allow users to review the clinical outcomes of their own patients, with or without a comparison to the clinical outcomes of other patients; and recommendation pages or displays that provide tips, guidance, and suggestions to improve the treatment plan, therapy, and medical outcome of the patient. These and other features and functions enable a patient coach to quickly and easily review and provide insights and related functions together in a single application.

For the sake of brevity, conventional features and functionality related to infusion systems, insulin pumps, infusion sets, and fluid reservoirs may not be described in detail here. Examples of infusion pumps and/or related pump drive systems used to administer insulin and other medications may be of the type described in, but not limited to, U.S. Pat. Nos. 5,505,709; 6,485,465; 6,554,798; 6,558,351; 6,659,980; 6,752,787; 6,817,990; 6,932,584; and 7,621,893; which are herein incorporated by reference.

As used herein, an “outcome” is a patient-related result having some correlation to medical treatment or therapy. For the exemplary embodiment described herein, a “glycemic outcome” is a patient-related result that is associated with the patient's glycemic state, diabetes therapy, insulin status, condition of the insulin infusion device, or the like. More specifically, a glycemic outcome can correspond to a status of blood sugar levels, such as high, low, variable, in range, etc., and/or to a test score or value that is indicative of glycemic health (such as the commonly used A1C value).

As used herein, an “insight” is a statistically derived association between an action/event (or a collection of actions/events) and a corresponding outcome, or an observation of a particular pattern of interest, which may be a device usage insight. In this regard, a “glycemic insight” is a statistically derived association between an action/event (or a collection of actions/events) and a corresponding outcome as measured by glucose readings.

In certain embodiments, the cloud-based system executes a plurality of insight detection algorithms when reviewing patient data. There can be any number of detectable insights and, consequently, any number of independently executing insight detection algorithms. Detectable insights may include any of the following, without limitation: increased time in target glucose range; increased time using automated insulin delivery mode; Xnumber of days since the last sensor use, e.g., seven days; temporary loss of sensor sensitivity; poor sensor accuracy; frequent exits from automated insulin delivery mode. The various insight detection algorithms may run as soon as a patient, a coach, a healthcare professional (HCP), or a caregiver uploads the patient's data to the system.

The system may prioritize the delivery of insight messages among different patients and for individual patients. For example, once the insights are detected for a population of patients, they can be ranked (per patient and/or among the population of different patients). The ranking enables the system to prioritize the delivery of insight messages or notifications based on predetermined criteria. In accordance with certain embodiments, insight and patient priority considers the following factors, without limitation: patient safety; patient burden; likelihood of attrition; insight frequency; and timeliness of insights. High priority can be assigned to patients with safety observations, medium priority can be assigned to patients with attrition observations, and low priority can be assigned to other patients. High priority patients are contacted within a relatively short period of time (e.g., within 24 or 48 hours), while low priority patients can be addressed in accordance with pre-planned touchpoints or a predetermined schedule.

As mentioned above, patient representatives (such as HCPs, coaches, parents, or the like) can access patient data and insights by logging into a suitably formatted and provisioned website or web-based portal. A representative's view includes a prioritized list of their assigned patients. Representatives can also search for patients that may not appear on their list. The online application (website) can provide any or all of the following content, without limitation: data summarizes; detailed glucose plots or graphs derived from continuous glucose sensor data; and timelines and detected insights for individual patients. In certain embodiments, the online application also supports user feedback and input. For example, the application may allow patient coaches or caregivers to mark insights once they have addressed them with the patient. Alternatively or additionally, the system can obtain feedback information from other data sources for use with its prioritization logic. The application can use this feedback, along with touchpoints logged in other data management systems, to adjust the insight and patient priority accordingly after an insight has been discussed with the patient. Moreover, insight messages may include features, active elements, or functionality to collect user feedback. For example, an insight message may include an active link, selectable buttons, and/or questions intended for the patient. User responses and actions can be monitored or recorded, and fed back to the cloud-based system for storage and ongoing analysis. In this way, interactive patient responses to coaching can be utilized to modify, enhance, or otherwise steer the coaching program to better suit the current needs of the patient.

In accordance with certain use cases, the cloud-based system described here provides coaching, guidance, advice, and recommendations that are intended to help patients meet certain behavioral goals that relate to the proper and effective use of their therapy delivery devices, e.g., insulin infusion pumps and/or continuous glucose sensors. Although the system can generate and present glycemic insight messages that are intended to improve the patient's glycemic outcome, the exemplary embodiment presented here is focused more on coaching, educational, and training aspects. For example, the system can provide coaching insight messages that serve as an extension of the native “user manual” or “help settings” that may be hard coded into the patient's medical device. Such coaching insights can provide additional instructions and tutorials related to the proper use of the medical device and/or related to customizations or adjustments that might be needed to address patient-specific goals or objectives. Such coaching insights may relate to various actions or activity including, without limitation: sensor maintenance and calibration tips; infusion pump maintenance tips; recommendations intended to increase the lifespan of a glucose sensor; recommendations to improve the accuracy of a glucose sensor; recommendations related to better placement/deployment of a glucose sensor on the skin of the patient; etc.

Some coaching insight messages can be presented for viewing and consideration by a patient representative (a coach) and/or an HCP, without notification to the patient. For example, some medical device settings should not be changed by patients. Accordingly, the patient's doctor can be provided with certain insight messages that require approval. If the doctor approves a recommended adjustment, then appropriate instructions can be delivered to the medical device for automated implementation of the approved adjustment. To this end, an HCP can log into and navigate the website maintained by the coaching platform, view the recommended changes, and approve the changes.

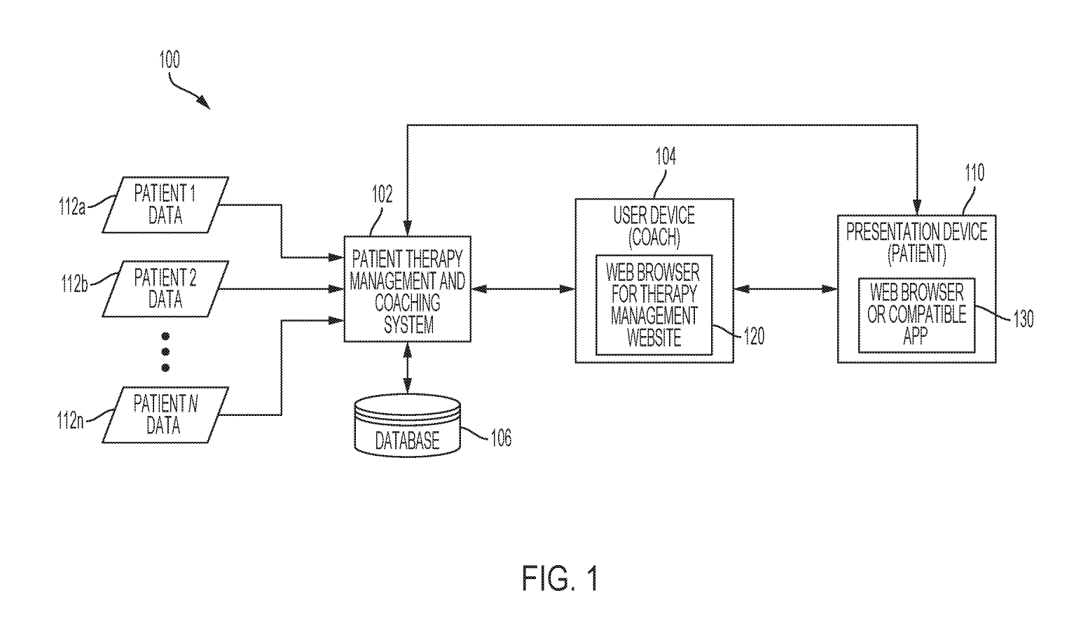

Turning now to the drawings, FIG. 1 is a simplified block diagram representation of an operating environment 100 that is suitably configured to support the techniques and methodologies described in more detail below. The operating environment 100 supports users of insulin infusion devices, and supports various techniques and methods to help end users (patient representatives, coaches, caregivers, healthcare providers, parents, etc.) manage, coach, and provide training in the use of insulin infusion devices. It should be appreciated that FIG. 1 depicts one possible implementation of the operating environment 100, and that other arrangements, architectures, and deployments can be provided if so desired. The operating environment 100 (which has been simplified for purposes of illustration) generally includes or cooperates with the following components, without limitation: a cloud-based patient therapy management and coaching system 102; a user device 104; and a database system 106 associated with the management and coaching system 102. The operating environment 100 may also include or support at least one presentation device 110 that is owned, operated, or used by the patient. In a typical deployment, the operating environment 100 supports many different user devices 104 and many different presentation devices 110, such that a centralized management and coaching system 102 can service a population of end users.

The management and coaching system 102 and the user device 104 are communicatively coupled to a data communication network (not shown). In certain embodiments, the user device 104 communicates with the presentation device 110, directly or via the data communication network. In practice, the operating environment 100 may cooperate with and leverage any number of wireless and any number of wired data communication networks maintained or operated by various entities and providers. Accordingly, communication between the various components shown in FIG. 1 may involve multiple network links and different data communication protocols. In this regard, a network utilized in the operating environment 100 can include or cooperate with any of the following, without limitation: a local area network; a wide area network; the Internet; a personal area network; a cellular communication network; a satellite communication network; a video services or television broadcasting network; a network onboard a vehicle; or the like. To this end, hardware components in the operating environment 100 may be suitably configured to support a variety of wireless and wired data communication protocols, technologies, and techniques as needed for compatibility with the network infrastructure.

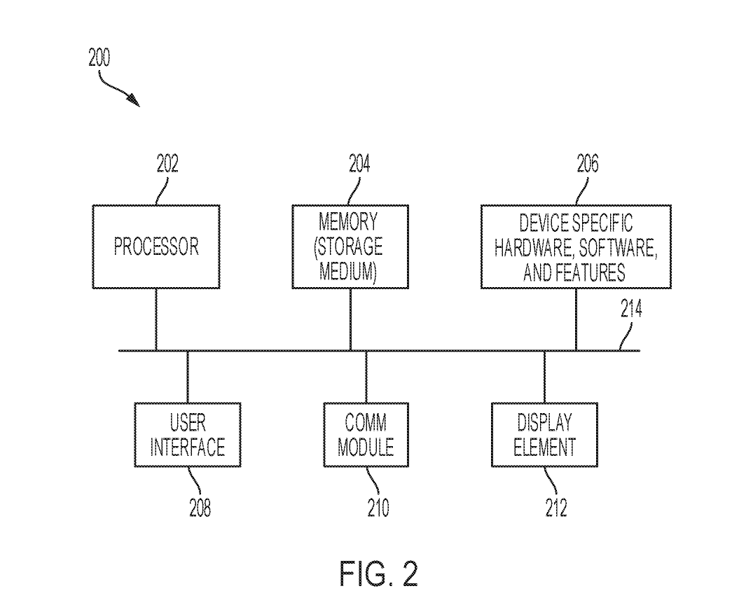

In accordance with certain exemplary embodiments, the management and coaching system 102 is implemented as at least one computer-based or processor-based component. For simplicity and ease of illustration, FIG. 1 depicts the system 102 as a single block—it should be appreciated that any number of distinct hardware components can be utilized to implement the system 102. An exemplary embodiment of a device suitable for implementing the system 102 is described below with reference to FIG. 2.

The management and coaching system 102 can be considered the “heart” of the operating environment 100. The system 102 includes or cooperates with the database system 106 (which is realized using one or more components) to support the functionality and operation described in more detail below. The system 102 collects and analyzes patient data 112 for a plurality of different patients, typically a very large population of patients under the care of many different caregivers. The database system 106 (which may be implemented as a generalized distributed file system) collects, stores, and maintains the patient data for the population of patients. In this regard, FIG. 1 depicts patient data 112a associated with Patient 1, patient data 112b associated with Patient 2, and so on, including patient data 112n associated with Patient N, where N can be any number of different patients. The patient data for any one patient can originate from various sources, including, without limitation: an insulin infusion device; a continuous glucose sensor; a blood glucose meter; a smartphone or other type of personal mobile device; a computing device; an activity tracker; a meal logging device or application; a mood tracking device or application; a GPS device; a vehicle owned or operated by the patient; a wearable smart device; a smart home controller system; a video game system; home entertainment equipment; etc. The system 102 can receive any or all of the patient data directly from one or more of these data sources. Alternatively or additionally, the system 102 can receive any or all of the patient data indirectly by way of a suitably configured data uploader component (not shown), which in turn receives the patient data from one or more of the originating sources.

The user device 104 is a client device that is owned or operated by the user, e.g., a patient coach, patient representative, an HCP, a caregiver, a nurse, a doctor, or the like. This description assumes that a patient coach owns/operates the user device 104, and that the information presented at the user device 104 need not be (and typically is not) intended for direct viewing by the patient. That said, the subject matter and methodology described here is not limited to such an implementation, and the user device 104 in certain scenarios may be owned/operated by the patient.

In certain embodiments, some or all of the functionality and processing intelligence of the management and coaching system 102 can reside at the user device 104. In other words, the operating environment 100 need not rely on a network-based or a cloud-based server arrangement, although such a deployment might be the most efficient and economical implementation. In other embodiments, some or all of the functionality and processing intelligence of the system 102 can reside at the presentation device 110, and/or at other compatible components or computing devices. These and other alternative arrangements are contemplated by this disclosure. To this end, some embodiments of the operating environment 100 may include additional devices and components that serve as data sources, data processing units, and/or content delivery mechanisms. For example, the operating environment 100 may include any or all of the following elements, without limitation: computer devices or systems; patient monitors; healthcare provider systems; data communication devices; and the like.

In accordance with certain exemplary embodiments, each user device 104 supported by the system 102 is implemented as a computer-based or processor-based component. For simplicity and ease of illustration, FIG. 1 depicts only one user device 104. In practice, however, the system 102 is suitably configured to support a plurality of user devices 104 (for example, to support a plurality of different patient coaches). An exemplary embodiment of a device suitable for implementing the user device 104 is described below with reference to FIG. 2; the user device 104 can be realized using a variety of different device platforms. For example, the user device 104 can be implemented as any of the following, without limitation: a cellular telephone or smartphone; a portable computer (e.g., a laptop, a tablet, or a netbook computer); a portable media player; a portable video game device; a portable medical device; a navigation device such as a global positioning system (GPS) device; a wearable computing device; an electronic toy or game; etc.

The remainder of this description assumes that the user device 104 is a computer device (a desktop computer, a laptop computer, a tablet device, a mobile device, etc.) used by a particular patient coach. To this end, the configuration and general functionality of the user device 104 can be substantially consistent with conventional personal computer design. In this regard, a web browser application 120 is installed on the user device 104 to allow the coach to access and utilize a suitably configured and designed therapy management and coaching website that is maintained and provided by the system 102. The management and coaching website allows the coach to organize and view patient data, obtain therapy recommendations based on patient data, and receive content related to patient therapy (e.g., messages, notifications, recommendations, instructions, and guidance) as generated by the system 102. In certain embodiments, the web browser application 120 and the associated website can be manipulated to upload patient data to the system 102 for storage and analysis.

The presentation device 110 is a client device that is owned or operated by the patient. In certain embodiments, some or all of the functionality and processing intelligence of the management and coaching system 102 can reside at the presentation device 110. In accordance with certain exemplary embodiments, each presentation device 110 is implemented as a computer-based or processor-based component. For simplicity and ease of illustration, FIG. 1 depicts only one presentation device 110. In practice, however, any number of distinct presentation devices 110 can be in data communication with the user device 104 and/or the system 102. An exemplary embodiment of a device suitable for implementing the presentation device 110 is described below with reference to FIG. 2. The presentation device 110 can be realized using a variety of different device platforms, including any of the platforms listed above for the user device 104.

The remainder of this description assumes that the presentation device 110 is a computer device (a desktop computer, a laptop computer, a tablet device, a mobile device, etc.) used by the particular patient. To this end, the configuration and general functionality of the presentation device 110 can be substantially consistent with conventional personal computer design. The presentation device 110 includes suitably configured applications, software, and features that support the display/presentation of information and content to the user, and that support the capture and transmission of patient-entered data, information, files, and the like. For example, a web browser application 130 (or a suitably configured and compatible application) is installed on the presentation device 110 to allow the patient to access and utilize a suitably configured and designed patient portal website that is maintained and provided by the management and coaching system 102. The patient portal website allows the patient to organize and view patient data, obtain therapy recommendations based on patient data, and receive content related to patient therapy (e.g., messages, notifications, recommendations, instructions, and guidance) as generated by the system 102. In certain embodiments, the web browser application 130 and the associated therapy management website can be manipulated to upload individual patient data to the system 102 for storage and analysis. In practice, the data and statistics from the system 102 for the user device 104 and the presentation device 110 can be synchronized to avoid conflicts between the user device 104 and the presentation device 110.

As mentioned above, the operating environment 100 includes or cooperates with computer-based and/or processor-based components having suitably configured hardware and software written to perform the functions and methods needed to support the features described herein. For example, the management and coaching system 102, each user device 104, and each presentation device 110 can be realized as an electronic processor-based component. In this regard, FIG. 2 is a simplified block diagram representation of an exemplary embodiment of a computer-based or processor-based device 200 that is suitable for deployment in the system shown in FIG. 1.

The illustrated embodiment of the device 200 is intended to be a high-level and generic representation of one suitable platform. In this regard, any of the computer-based or processor-based components mentioned here can utilize the architecture of the device 200. The illustrated embodiment of the device 200 generally includes, without limitation: at least one processor 202; a suitable amount of memory 204; device-specific hardware, software, firmware, and/or features 206; a user interface 208; a communication module 210; and a display element 212. Of course, an implementation of the device 200 may include additional elements, components, modules, and functionality configured to support various features that are unrelated to the subject matter described here. For example, the device 200 may include certain features and elements to support conventional functions that might be related to the particular implementation and deployment of the device 200. In practice, the elements of the device 200 may be coupled together via a bus or any suitable interconnection architecture 214.

The processor 202 may be implemented or performed with a general purpose processor, a content addressable memory, a digital signal processor, an application specific integrated circuit, a field programmable gate array, any suitable programmable logic device, discrete gate or transistor logic, discrete hardware components, or any combination designed to perform the functions described here. Moreover, the processor 202 may be implemented as a combination of computing devices, e.g., a combination of a digital signal processor and a microprocessor, a plurality of microprocessors, one or more microprocessors in conjunction with a digital signal processor core, or any other such configuration.

The memory 204 may be realized as RAM memory, flash memory, EPROM memory, EEPROM memory, registers, a hard disk, a removable disk, a CD-ROM, or any other form of storage medium known in the art. In this regard, the memory 204 can be coupled to the processor 202 such that the processor 202 can read information from, and write information to, the memory 204. In the alternative, the memory 204 may be integral to the processor 202. As an example, the processor 202 and the memory 204 may reside in an ASIC. At least a portion of the memory 204 can be realized as a computer storage medium, e.g., a tangible computer-readable medium having computer-executable instructions stored thereon. The computer-executable instructions, when read and executed by the processor 202, cause the device 200 to perform certain tasks, operations, functions, and processes that are specific to the particular embodiment. In this regard, the memory 204 may represent one suitable implementation of such computer-readable media. Alternatively or additionally, the device 200 could receive and cooperate with computer-readable media (not separately shown) that is realized as a portable or mobile component or platform, e.g., a portable hard drive, a USB flash drive, an optical disc, or the like.

The device-specific hardware, software, firmware, and features 206 may vary from one embodiment of the device 200 to another. For example, the device-specific hardware, software, firmware, and features 206 will support: smartphone functions and features when the device 200 is realized as a mobile telephone; conventional personal computer functions and features if the device 200 is realized as a laptop or tablet computer; insulin pump operations when the device 200 is realized as an insulin infusion device; etc. In practice, certain portions or aspects of the device-specific hardware, software, firmware, and features 206 may be implemented in one or more of the other blocks depicted in FIG. 2.

The user interface 208 may include or cooperate with various features to allow a user to interact with the device 200. Accordingly, the user interface 208 may include various human-to-machine interfaces, e.g., a keypad, keys, a keyboard, buttons, switches, knobs, a touchpad, a joystick, a pointing device, a virtual writing tablet, a touch screen, a microphone, or any device, component, or function that enables the user to select options, input information, or otherwise control the operation of the device 200. The user interface 208 may include one or more graphical user interface (GUI) control elements that enable a user to manipulate or otherwise interact with an application via the display element 212.

The communication module 210 facilitates data communication between the device 200 and other components as needed during the operation of the device 200. In the context of this description, the communication module 210 can be employed to transmit or stream device-related control data, patient data, device-related status or operational data, messages and notifications, therapy related content, and the like. It should be appreciated that the particular configuration and functionality of the communication module 210 can vary depending on the hardware platform and specific implementation of the device 200. In practice, an embodiment of the device 200 may support wireless data communication and/or wired data communication, using various data communication protocols. For example, the communication module 210 could support one or more wireless data communication protocols, techniques, or methodologies, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; cellular/wireless/cordless telecommunication protocols; wireless home network communication protocols; paging network protocols; magnetic induction; satellite data communication protocols; wireless hospital or health care facility network protocols such as those operating in the WMTS bands; GPRS; and proprietary wireless data communication protocols such as variants of Wireless USB. Moreover, the communication module 210 could support one or more wired/cabled data communication protocols, including, without limitation: Ethernet; powerline; home network communication protocols; USB; IEEE 1394 (Firewire); hospital network communication protocols; and proprietary data communication protocols.

The display element 212 is suitably configured to enable the device 200 to render and display various screens, recommendation messages, notifications, GUIs, GUI control elements, drop down menus, auto-fill fields, text entry fields, message fields, or the like. Of course, the display element 212 may also be utilized for the display of other information during the operation of the device 200, as is well understood. Notably, the specific configuration, operating characteristics, size, resolution, and functionality of the display element 212 can vary depending upon the practical implementation of the device 200. For example, if the device 200 is a laptop computer, then the display element 212 may be a relatively large monitor. Alternatively, if the device 200 is a cellular telephone device, then the display element 212 may be a relatively small integrated display screen, such as a touch-sensitive screen.

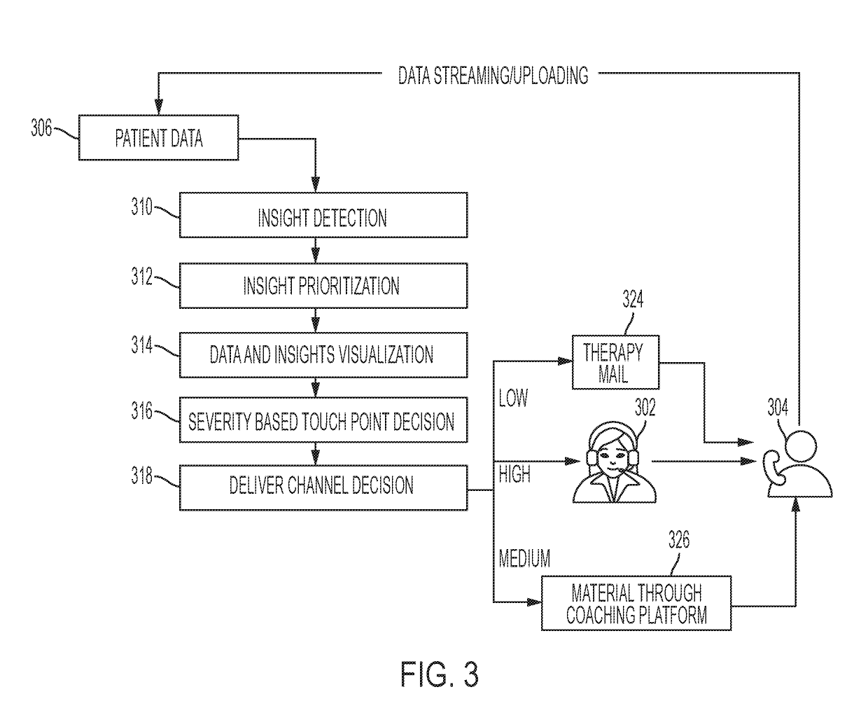

FIG. 3 is a diagram that illustrates a typical use case that can be supported by the system shown in FIG. 1. For the illustrated example, a coach 302 (e.g., a customer support agent, a clinical specialist, a diabetes educator, a doctor, or a nurse) is assigned to one or more patients 304. Patient data 306 associated with the patients 304 is streamed, uploaded, or otherwise communicated to the centralized management and coaching system, which is not separately depicted in FIG. 3. The uploaded patient data is subjected to various routines, procedures, algorithms, and/or processes to obtain useful and insightful information related to the management of therapy and behavioral coaching of the patients 304.

In accordance with the illustrated embodiment, the uploaded patient data is subjected to an insight detection process 310 to determine whether an insight message should be generated for any given patient. This description assumes that a number of insight messages are generated. The insight messages are subjected to an insight prioritization process 312, which prioritizes/ranks the insight messages based on predetermined criteria (as described above). The insight messages to be delivered are visualized through a data and insights visualization process 314. This includes, but is not limited to illustrations describing event background, event patterns, correlations, and comparative analyses. The illustrated insight is then assigned to the insight pipeline workflow based on a severity based touch point decision process 316, which sorts the upcoming touchpoints by priority (based on urgency, severity, patient history, etc.). A deliver channel decision process 318 determines through which platform the insight will be delivered to patients, an app, or web application, with or without the coach's discretion.

Relatively low priority insight messages can be communicated to the patients 304 by way of therapy mail 324. In this context, therapy mail 324 refers to an email outside of the application that has detail of insight sentence and illustration, as opposed to the application where a real time notification will be sent. Relatively medium priority insight messages can be communicated to the patients 304 by way of the coaching platform 326 that is described in more detail herein. Relatively high priority insight messages can be quickly communicated to the patients 304 by the coach 302, via an automated telephone call, an automated email, a text message, or the like. Thus, the output and/or results of the processing and analysis performed by the management and coaching system can be used to determine how best to communicate with the patients 304, when to deliver insight messages to the patients 304, and the like. Moreover, the output and/or results of the processing and analysis performed by the management and coaching system can be leveraged in the manner described below to provide patient coaching/training programs by way of the coaching platform 326. In practice, the coach 302, the therapy mail 324, and the coaching platform 326 work together to help the patient achieve better outcomes. These aspects are complementary to each other.

The management and coaching system 102 collects and analyzes a large amount of patient data to generate insight messages that are used in connection with patient coaching, training, and education. The patient data can be processed and analyzed using, for example: machine learning algorithm(s); expert system technology; artificial intelligence techniques; a knowledge base; natural language processing; and/or similar methodologies. The output generated by the system 102 enables patient coaches to monitor patient progress in meeting certain behavioral and/or clinical outcome goals. The system 102 can generate useful output (graphs, charts, reports, notifications, statistics related to patient behavior, statistics related to medical device usage or performance, statistics related to medical device alarms or alerts, and so on) that are intended to benefit the coaches and the patients. In certain implementations, the system 102 utilizes the data analytics and insight delivery system and methodologies disclosed in United States Patent Application Publication number US 2017/0053072, the content of which is incorporated by reference herein.

In preferred implementations, the system 102 generates, maintains, and updates a web portal or website that is intended for use by patient coaches. Thus, a coach can conveniently log into the web portal using his or her credentials to access the various features and functionality described here. In practice, the web portal can be accessed using any device that is web-enabled, browser-based, HTTP compatible, or the like. In this way, a patient coach using the management and coaching system 102 can quickly and conveniently determine whether or not the patients assigned to her are progressing in their designated goals.

In practice, the system 102 requires a minimum amount of input data per patient before it can generate intelligent and accurate results. For example, it may be necessary to collect patient data for at least one full day. Going forward, however, the information output by the system 102 will progressively become more sophisticated and accurate as more and more patient data is collected and analyzed. In this regard, the techniques and methodologies described herein assume that the sources of input data (such as glucose sensors, blood glucose meters, physiological sensors, and the like) are each operating within an acceptable range of accuracy.

In certain embodiments, the system 102 obtains some patient data in the form of mobile device data provided by the patient's mobile device. The mobile device data can include any type of data or information generated by the mobile device, forwarded by the mobile device, entered at the mobile device, detected by the mobile device, or the like. For example, and without limitation, the mobile device data can include time stamp data, calendar data, mobile app data, status information related to the operation of the mobile device, and/or sensor data generated by sensors or detectors onboard the mobile device (such as an accelerometer, a gyroscope, a light sensor, a camera, a thermometer, a biometric scanner, etc.).

Data Inputs

There are many factors that can influence a patient's blood glucose levels. Various factors may also influence how best to control and manage the patient's blood glucose. The glycemic insights methodology presented here is based on the collection and analysis of data, which need not be specifically related to blood glucose (BG) meter measurements, glucose sensor readings, or insulin delivery information. Although the system 102 obtains and analyzes such data, it may also obtain and consider additional data. The system 102 may also process data received directly or indirectly from other physiological sensors, devices, or equipment. For example, an embodiment of the system 102 can be suitably configured to analyze respiratory data, electrocardiogram data, body temperature data, heartrate information, and the like.

The system 102 is suitably configured to receive and process a variety of input data from multiple sources. Moreover, the system 102 is designed to be flexible and scalable to accommodate additional input data types as needed. The number of input data sources and the amount of input data handled by the system 102 may vary from one embodiment to another, depending on the particular implementation and intended application. In accordance with the embodiment described here, some or all of the following input data can be used for purposes of generating coaching-related output. The following summary of specific input data types is not intended to be exhaustive or otherwise limiting, and alternative or additional input data can be considered in an embodiment of the system 102.

Carbohydrate amount—this refers to the carbohydrate amount that one Unit of insulin can compensate to maintain the current glucose level. The carbohydrate amount is usually expressed in grams or milligrams. The patient's mobile device will usually be the source of this data.

Bolus information—the bolus information includes the bolus dosage amount (in Units of insulin), the date/time of delivery (time of day and calendar data), and the bolus type (normal, square, or dual). The insulin infusion device will usually be the source of this data.

Insulin to carbohydrate ratio—this is a patient-specific parameter that relates to how much insulin the patient needs to compensate for a designated unit (e.g., one gram) of carbohydrate. The insulin to carbohydrate ratio is expressed in grams/Unit. The insulin infusion device will usually be the source of this data.

Insulin sensitivity factor—this is a patient-specific parameter that relates to the reduction in blood glucose in response to one Unit of insulin. The particular manner in which the insulin sensitivity factor is calculated is determined by the specific pumping protocol. The insulin sensitivity factor is expressed in mg/dL/U (milligrams per deciliter per Unit). The insulin infusion device will usually be the source of this data.

Active insulin amount—this refers to how much insulin is still active in the body of the patient from previous bolus doses. This quantity is expressed in Units of insulin. The insulin infusion device will usually be the source of this data.

Time of day—this refers to timestamp and/or date stamp information, which may be associated with or appended to any other piece of input data to provide a time reference.

Basal rate—this is a patient-specific parameter that indicates the basal rate of insulin delivery, which is usually expressed in Units/hour. The insulin infusion device will usually be the source of this data.

Temporary basal use—this refers to an occurrence during which the patient temporarily “overrides” the nominal or usual basal rate of insulin. The system employs a Boolean value that indicates the activation of the temporary basal mode, and also indicates the temporary basal rate value. The insulin infusion device will usually be the source of this data.

Consecutive boluses—this refers to an occurrence of back-to-back insulin boluses, which are delivered within a designated period of time. The system employs a Boolean value that indicates the occurrence of consecutive boluses, and also indicates the total volume of the boluses delivered during the designated period of time. The insulin infusion device will usually be the source of this data.

Insulin suspension—this refers to a period of time during which the insulin infusion device has been temporarily suspended (insulin delivery is temporarily halted). The data related to insulin suspension can include some or all of the following, without limitation: threshold setting; suspension duration; active insulin before the suspension; sensor rate of change around the suspension; carbohydrate intake around the suspension; time (day of week, time of day) of the suspension; how the suspension recovered; and user response to the suspension. The insulin infusion device will usually be the source of this data.

Reservoir rewind and priming time—this refers to activities associated with the installation of a new insulin reservoir into the insulin infusion device. This requires a rewind action to retract the reservoir actuator, which facilitates removal of the used reservoir. After installing the new reservoir, the fluid flow path is primed for insulin delivery. The insulin infusion device will usually be the source of this data.

Pump alarms and associated alarm times—pump alarms can be generated by the insulin infusion device for various reasons. Pump alarm data indicates the type of alarm and the corresponding alarm time. The insulin infusion device will usually be the source of this data.

Sensor alerts and alert times—sensor alerts can be generated by the insulin infusion device and/or the glucose sensor for various reasons. Sensor alert data indicates the type of sensor alert and the corresponding alert time. The insulin infusion device and/or the glucose sensor can be the source of this data.

Blood glucose readings and measurement times—blood glucose readings are usually expressed in mg/dl, and are obtained from a blood glucose meter. The insulin infusion device, the blood glucose meter, or the patient's mobile device can be the source of this data.

User demographic information—this data may include, without limitation, the patient's age, number of years using insulin, medical diagnosis, age at the onset of diabetes, sex, medication types, and the like. User demographic information can be provided by the patient's mobile device, the insulin infusion device, a webpage user interface, or the like.

Meal time and content—this data relates to the timing of meal consumption and the type and amount of food consumed. The patient's mobile device will usually be the source of this data. In this regard, a suitably configured mobile app can include a feature or functionality that allows the patient to specify meal times and to estimate the type and amount of food consumed at each meal. In certain scenarios, this data can be imported from a third party (partner) database directly, rather than having patients redundantly enter the information into the mobile app.

Exercise time and content—this data relates to the timing of exercise and the type, duration, and amount of exercise performed by the patient. The patient's mobile device or an activity tracker device will usually be the source of this data. In this regard, a suitably configured mobile app can include a feature or functionality that allows the patient to specify exercise times and to estimate the type and amount of exercise. In certain scenarios, this data can be imported from a third party (partner) database directly, rather than having patients redundantly enter the information into the mobile app.

Medication type, dosage, and time—this data relates to instances when the patient takes medication (other than insulin), and the data indicates the type of medication, the dosage taken, and the time that the medication was taken. The patient's mobile device will usually be the source of this data. In some scenarios, a smart insulin pen or other type of smart insulin delivery device can be the source of this data. In this regard, a suitably configured mobile app can include a feature or functionality that allows the patient to record information associated with taking medication.

Sleep time and quality—this data indicates sleeping periods, and information related to the quality or type of sleep experienced by the patient. The sleep-related information can be provided by a patient monitor (activity tracker) or, in certain embodiments, the sleep-related information is provided by a suitably configured mobile app running on the patient's mobile device. In such an embodiment, the mobile app allows the patient to enter the relevant sleep-related information. In accordance with some embodiments, sleep related information can be calculated using accelerometer data, heartrate data, ambient lighting measurements, glucose levels, etc.

Stress time—this data indicates periods of stress experienced by the patient. The stress-related information can be derived from physiological factors and/or measurable data such as heart rate, blood pressure, skin conductance, body temperature, or the like. Additionally or alternatively, the stress-related information can be based on user input. Accordingly, the patient's mobile device can be the source of this data. A suitably configured mobile app can include a feature or functionality that allows the patient to record information associated with periods of stress.

Electronic medical records and lab test data—this data can be provided by healthcare providers, medical facilities, insurance companies, or the like. In certain scenarios, this data can be imported from a third party (partner) database directly, rather than having patients redundantly enter the information into the mobile app.

As mentioned above, the patient management and coaching system 102 can be utilized to provide personalized coaching, training, and education to a patient, with interactive assistance from a coach assigned to that patient. The system 102 performs certain automated tasks related to the review and analysis of patient data, the processing of patient input and feedback, and delivery of coaching related information to the coach and/or the patient. In accordance with the exemplary use case described here, each coaching program for a patient is broken down into three primary stages or milestones: (1) the onboarding stage; (2) the tracking stage; and (3) the final review stage. Each coaching program can be customized based on the patient's needs, objectives, requests, goals, or the like. At the outset, the patient can identify her objectives and goals for the coaching program, and the coaching system 102 automatically determines how best to arrange and configure the coaching program based on the patient-identified objectives and goals. Based on the identified goal(s), the coaching system 102 can select or utilize one or more insight engines, algorithms, or modules that best fit the patient's needs at that time. Over time, the coaching system 102 monitors patient progress and performance by collecting and analyzing updated patient data to determine whether or not new, additional, or modified coaching or training might be needed. At the end of the coaching program, the coach can review the results with the patient to determine whether to continue with another coaching program, to extend the current program, to add one or more parallel coaching programs, or the like.

At the onboarding stage, the patient indicates the goals, objectives, problems, questions, or desired results of the coaching program (hereinafter simply referred to as “goals”). The goals can be identified by the patient with or without the assistance of an HCP, a diabetic trainer, a coach, etc. In certain embodiments, the management and coaching system 102 accommodates the entry of any number of goals. In alternative embodiments, the coaching system 102 utilizes a library of common goals (and sub-goals) that can be selected by the patients. The following are several examples of common patient goals; this list of goals is not intended to be all-inclusive or limiting of the scope or application of the coaching system 102 in any way. Exemplary goals include: I want to minimize the time it takes to recover from a low glucose event; I want sleep better; I want to eat whatever I want to eat; I want to accurately configure my insulin pump settings; I want to make sure my baby is healthy.

At the onboarding stage, the management and coaching system 102 breaks down high level or generalized goals into low level or more specific goals (also referred to herein as “sub-goals”). More specifically, each high level goal can be broken down to any number of behavior goals and any number of outcome goals. The coach evaluates the current status of the patient based on existing data and communication with the patient, reaches an agreement with the patient, and confirms (to the management and coaching system 102) which goals (primary goals and sub-goals) are to be tracked. Each goal has its own coaching program timeframe that is designated by the management and coaching system 102.

The tracking stage allows the coach to use an online portal or dashboard as an interactive interface for purposes of tracking and monitoring the patient's progress. During the tracking stage, the patient can also be provided with an appropriate messaging/chatting platform to enable the patient to consume relevant content related to the coaching program, and to enable the patient to provide feedback to the coach and/or to the management and coaching system 102.

During the tracking stage, goal tracking insights are delivered to both patients and coaches periodically. Goal tracking insights evaluate whether the patient is exceeding, achieving, or missing the stated goal. Real-time observational insights are delivered to patients only. Observational insights are intended to correct or encourage certain patient behaviors. During the tracking stage, the management and coaching system 102 recommends educational/coaching insights, which can be reviewed by the coach and delivered to the patient at the coach's discretion.

During the tracking stage, the progression of training materials (and insight messages) is based on the following components, without limitation: explicit results from goal tracking insights; explicit natural language feedback from patients responding to real time observational insights; a ratio of occurrence of positive versus negative observational insights; and review of a patient journey timeline view, which indicates detailed events and content of related feedback.

At the final review stage, the management and coaching system 102 generates a final report that describes progress, achievements, challenges, opportunities, and recommendations for any subsequent coaching program(s), or the next stage of the current coaching program. During the final review stage, the coach can review the final reports with the patient and decide on the next steps. Recommendations related to the next coaching/training stage may be based on how the patient has performed in obtaining previous goals, based on recommendations obtained from the patient's HCP, and possibly other factors. In certain embodiments, the system evaluates the achievement for different levels of goals, and component scores and overall scores will both be generated.

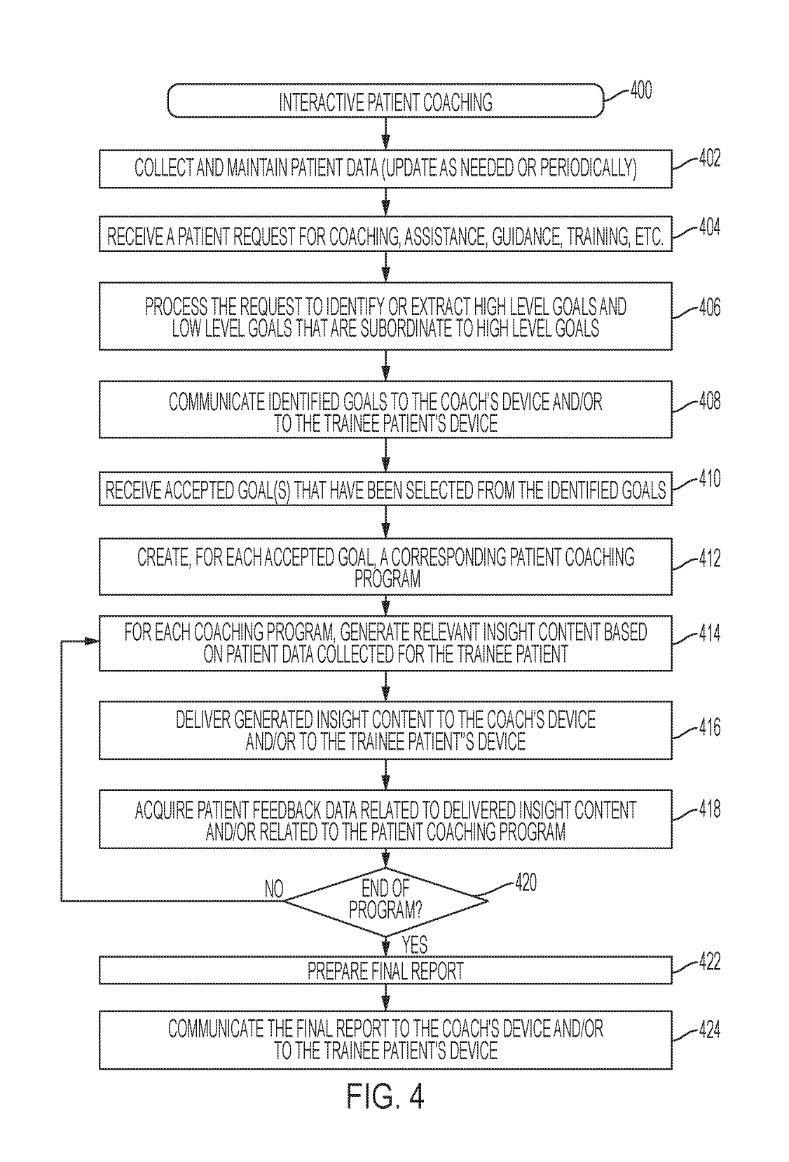

FIG. 4 is a flow chart that illustrates an exemplary embodiment of a process 400 for creating and managing interactive patient coaching programs. The various tasks performed in connection with the process 400 may be performed by software, hardware, firmware, or any combination thereof. For illustrative purposes, the following description of the process 400 may refer to elements mentioned above in connection with FIGS. 1-3. It should be appreciated that the process 400 may include any number of additional or alternative tasks, the tasks shown in FIG. 4 need not be performed in the illustrated order, and the process 400 may be incorporated into a more comprehensive procedure or process having additional functionality not described in detail herein. Moreover, one or more of the tasks shown in FIG. 4 could be omitted from an embodiment of the process 400 as long as the intended overall functionality remains intact.

The process 400 utilizes patient data that is associated with a population of different patients under the care of one or more HCPs. To this end, at task 402 the process 400 collects patient data that is associated with a plurality of medical device users (patients), such as diabetic patients that use insulin infusion pumps. Although the process 400 supports a plurality of different patients, the example described here relates to the creation, maintenance, and management of a coaching program for one particular patient, i.e., a trainee patient. Consequently, the trainee patient's HCP, the trainee patient, and the trainee patient's coach may be intended recipients of the output that results from the process 400.

The patient data can be obtained from the patients, from HCPs, directly from medical devices or other devices owned or operated by the patients/HCPs, indirectly via a data uploader device or system, or the like. The patient data can be obtained and updated in an ongoing manner, e.g., periodic data uploads, near real-time data transfer, manual data uploads, or the like. In accordance with the exemplary use case presented here, where the medical device users are insulin infusion device users, the patient data for any individual patient may include any or all of the following, without limitation: carbohydrate amount; bolus information; insulin to carbohydrate ratio; insulin sensitivity factor; active insulin amount; time of day; basal rate; temporary basal use; consecutive boluses; insulin suspension; reservoir rewind time; reservoir priming time; pump alarms and associated alarm times; sensor alerts and associated alert times; blood glucose readings and associated measurement times; user demographic information; meal times and corresponding meal content; exercise times and corresponding exercise content or type; medication type, dosage, and time; sleep time and quality; stress time; and electronic medical records; medical lab test data. Of course, the specific type and amount of data collected for each patient can vary from one implementation to another, depending on patient characteristics, medical device characteristics, the condition(s) being treated, HCP preferences, and other practical factors.

The process 400 receives a patient request for coaching, assistance, guidance, training, tutoring, support, or the like (task 404). In accordance with the exemplary embodiment presented here, the management and coaching system 102 receives and processes the patient request, which is usually generated by a computer-based device that is owned or operated by the trainee patient (referred to herein as the “patient device”). In this regard, the trainee patient can create and submit a patient request using any application, software, or feature of the patient device. For example, the patient request can be created and submitted with any of the following, without limitation: a suitably designed website; a specialized patient coaching application that is supported by the system 102; email; text messaging; instant messaging; a speech recognition application; a chat feature of a social media application; etc. As explained above, the system 102 may support a number of predefined goals that can be chosen by the trainee patient when creating the patient request. Alternatively or additionally, the system 102 may support free text entry by the trainee patient, such that the trainee patient can define her goal(s) in any desired manner.

Upon receiving a patient request, the system 102 processes the request to identify or extract one or more goals to be achieved by the trainee patient (task 406). The patient request may implicate any number of goals, including high level (general) goals and/or low level (specific) goals, each of which may subordinate to, or otherwise associated with, a respective high level goal. Moreover, the system 102 and the process 400 contemplate and support different types of goals, including, without limitation: patient outcome goals; and patient behavior goals. Task 406 can identify distinct goals that are conveyed in the patient request and determine whether an identified goal is an outcome goal or a behavior goal. For this particular example, where patients are diabetics using insulin therapy, a patient outcome goal relates to glucose control, glycemic health results, and/or overall patient health. In contrast, a patient behavior goal relates to patient action or activity, things that are within the control of the patient, patient reactions, and the like.

In certain implementations, the process 400 analyzes the patient request to identify any high level goals (and any distinguishable low level goals). The process 400 can “break down” each high level goal into corresponding low level goals, if applicable. Relative to a high level goal, each lower level goal is more specific, focused, and more well-defined. For example, the patient request may include the following goal, which is considered to be a high level goal: I want to sleep better. Associated lower level goals that may help achieve this goal include, without limitation: reduce medical device alerts and alarms that occur overnight; avoid hypoglycemic and hyperglycemic states overnight; avoid certain types of food before bedtime; and don't eat dinner less than three hours before bedtime. Notably, these lower level goals need not be explicitly identified or defined by the patient—the process 400 can determine and identify low level goals based on the respective high level goal that is explicitly identified in the patient request.

This example assumes that the system 102 identifies one or more goals from the patient request. The identified goals are communicated to, or are made accessible by, a computer-based user device that is owned, operated, or otherwise associated with the patient's coach (referred to herein as the “coach's device”) and/or are communicated to, or are made accessible by, the patient's device (task 408). For example, the identified goals can be presented to the coach/patient as a list of selectable items, using a suitably formatted webpage. As another example, the identified goals can be delivered to the coach/patient in an email, a text message, a chat application, a mobile application, or the like. In practice, the process 400 may provide a suitably configured therapy management and coaching website in a web browser running on the coach's computer device, wherein the website includes at least one webpage that includes information related to the coaching program and the trainee patient. A webpage, section, or component of this website can include an interactive or selectable list of the identified goals.

After consultation with the trainee patient, which may involve correspondence using the therapy management and coaching website, the coach obtains at least one accepted goal that has been selected from the identified goals. In this regard, the trainee patient and the coach can discuss the list of identified goals and come to an agreement regarding which (if any) of the identified goals will become the basis of a corresponding coaching program. To this end, the therapy management and coaching website can include a selectable list of the identified goals, to enable easy and convenient selection by the coach, the trainee patient, or both.

This description assumes that the system 102 receives at least one accepted goal to be assigned to the trainee patient (task 410). Thereafter, the system 102 creates an appropriate patient coaching program for each accepted goal (task 412). In practice, the system 102 creates a different coaching program for each accepted low level, system-defined goal. Consequently, the trainee patient may be subjected to a plurality of concurrent coaching programs, depending on the number of goals that have been accepted. A coaching program is associated with the delivery of insight messages, content, or information during a defined period of time (e.g., a month, eight weeks, three months, a year). The insight messages delivered to the coach and/or the trainee patient are based on at least some of the following, without limitation: automated computer-based review of the patient data; feedback received from the trainee patient; progress made by the trainee patient during the coaching program; satisfaction of milestone events; etc. Thus, a coaching program is highlighted by the insight messages, the timing of their delivery, and the manner in which the trainee patient responds to them.

In certain implementations, a coaching program includes three different types of insight messages: goal tracking insight messages that are intended for both the coach and the trainee patient; observational insight messages that are primarily intended for the trainee patient; and educational insight messages that are intended for delivery to the trainee patient at the discretion of the coach. Goal tracking insight messages generally indicate whether the trainee patient is meeting, exceeding, or not satisfying the goal of the coaching program. The system 102 generates goal tracking insight messages in response to an analysis of the patient data, such that the coach and the trainee patient can be notified of the progress toward meeting the stated goal. Observational insight messages provide encouragement, motivation, and/or corrective information to the trainee patient. This type of insight message can be generated and sent in real-time whenever deemed necessary by the system 102. Educational insight messages are initially delivered to the coach. The coach can approve the delivery or forwarding of an educational insight message to the trainee patient using, for example, a button, a link, or a user interface feature of the management and coaching website.

For each coaching program, the system 102 generates relevant insight messages/content as needed or as scheduled, based on the patient data collected for the trainee patient and based on additional coaching-related information obtained by the system 102 during the course of the coaching program (task 414). The generated insight messages are delivered to the coach's device and/or to the trainee patient's device as appropriate (task 416). Although most if not all insight messages will be delivered to the coach's device, only a limited number of insight messages may be delivered to the trainee patient's device. For this particular embodiment, goal tracking insight messages are delivered in accordance with a schedule that is specific to the patient coaching program. In contrast, observational insight messages are delivered on an as-needed basis, without regard to any predetermined schedule or timing constraints. Educational insight messages are delivered based on a review of delivered goal tracking insight messages and delivered observational insight messages. Thus, educational insight messages may be delivered in accordance with a schedule, periodically, or the like.

The process 400 may also acquire patient feedback data that is related to delivered insight messages and content and/or patient feedback data that is otherwise related to the patient coaching program (task 418). Such feedback can be entered and submitted by the trainee patient using the patient's device or any of the tools, features, and applications described above.

Patient data is updated and collected in an ongoing manner, and insight messages are generated and delivered throughout the duration of the coaching program (the “No” branch of query task 420). Upon completion of the patient coaching program (the “Yes” branch of query task 420), the system 102 prepares a final report (task 422) and communicates the report to the coach's device and/or to the trainee patient's device (task 424). The final report summarizes the results and outcome of the coaching program. The coach can review the final report with the trainee patient to determine how best to proceed. For example, if the stated goal was not successfully met, then the coaching program can be repeated with or without modifications. If the trainee patient met the goal, then a new coaching program with more advanced training or education can be initiated.

### Example 1

The following work flow example relates to a patient who is concerned about nocturnal hypoglycemia and related sleep issues.

Onboarding Stage:

The patient request indicates “I want to sleep better”

The system 102 breaks down this high level goal into three low level goals: (1) reaches glucose value between 100-120 mg/dl before sleep (an outcome goal); (2) reaches 30 consecutive days with no food/insulin within two hours before sleep (behavior goal); (3) reaches 30 consecutive days of calibrating the glucose sensor before sleep (behavior goal).

Tracking Stage:

The coach uses the website dashboard and timeline to track the progress of the patient. The system delivers three different levels of insight messages to patients. Insight message delivery is throttled by how the patient is engaged and the occurrence of late night hypoglycemic events. If not engaging, the system 102 will automatically push for more engagement insights. If not reducing the occurrence of hypoglycemic events, the system 102 will prompt the coach and escalate to HCPs if needed.

Final Review Stage:

The system 102 generates a final report describing whether the patient has been receiving any overnight alerts, has had any hypoglycemia event, has been able to consistently perform calibrations, and has avoided eating before sleep. The system 102 will recommend whether the patient needs a second session or can move on to the next struggling points (e.g., eat healthy at dinner) for the patient.

### Example 2

The following work flow example relates to a pregnant patient with gestational diabetes.

Onboarding Stage:

The patient request identifies: “I want my baby to be healthy”

The system 102 breaks down this high level goal into three low level goals: (1) reaches glucose value between 100-120 mg/dl three hours after a meal (outcome goal); (2) reaches monthly body weight growth between 1.0 to 1.5 kg (outcome goal); (3) eating correct nutrients in combination, as recommended by a nutrition insight recommendation (behavior goal).

Tracking Stage:

The coach uses the website dashboard and timeline to track the progress of the patient. Educational insights (nutrients and exercise) progress on a monthly basis according to the trimesters. If not eating healthy, engagement insight messages will be delivered more frequently and escalated to the coach. If weight is not adequately under control each month, nutrients and exercise insight messages will be adjusted to be delivered with different timing and/or with different content.

Final Review Stage:

The system 102 generates final reports describing whether the patient has been able to control her body weight growth, and whether she has been able to control her glucose levels. The system 102 will recommend postnatal glucose control training program as the next phase coaching program.

Patient Timeline Report

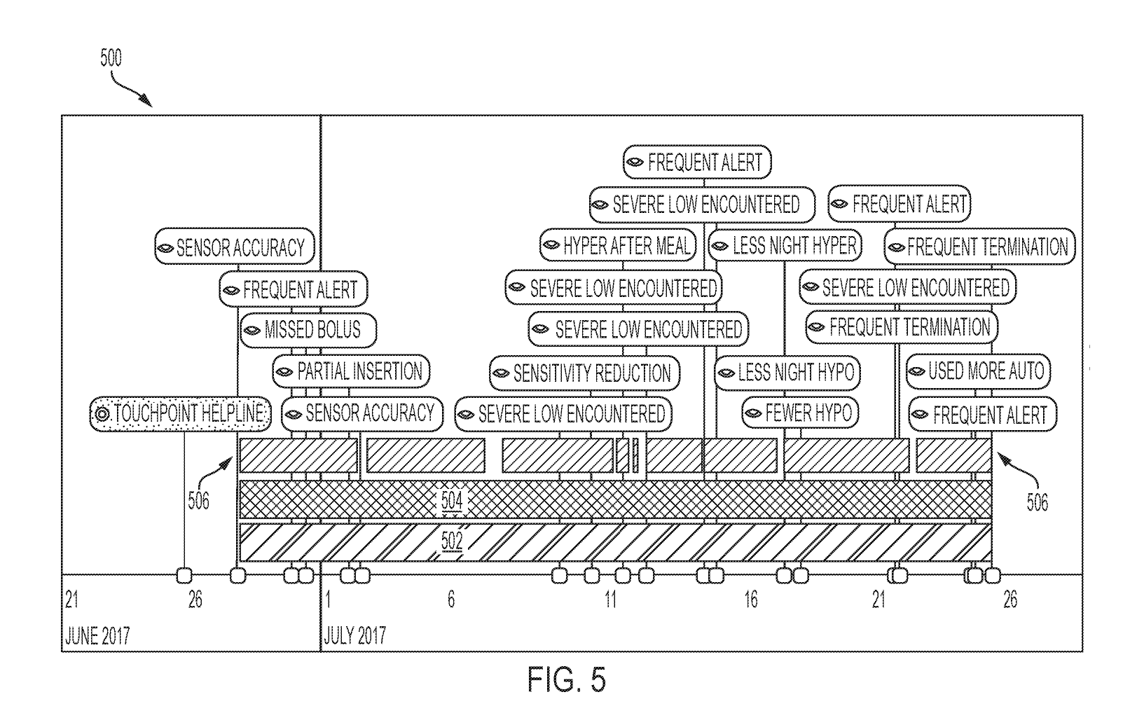

FIG. 5 is a diagram of a patient timeline 500. The actual content, format, and amount of information contained in the patient timeline 500 will vary from patient to patient, and from one time period to another time period for a given patient. The patient timeline 500 can be presented to the coach and/or to the trainee patient in connection with the delivery of an insight message, as a part of the final report, and/or at other times during the course of a coaching program. The horizontal scale represents passage of time. The shaded segment 502 indicates time during which the patient was wearing/using an insulin infusion pump. The shaded segment 504 indicates time during which the patient was wearing/using a continuous glucose sensor. The shaded segments 506 indicate periods of time during which the automated insulin delivery mode was active for the patient's insulin infusion pump. Although not shown in FIG. 5, the system can generate additional and/or alternative segments that run along the timeline if needed or desired to indicate other characteristics, behaviors, or aspects related to the patient's use of the medical device(s).

The patient timeline 500 includes a touchpoint marker 510 that indicates a time when the patient contacted a helpline. In practice, a patient timeline can include any number of touchpoint markers to graphically depict points in time when the patient reached out for assistance or otherwise made contact with an outside representative, a vendor, a support desk, or the like.

The patient timeline 500 also includes many observation markers, which appear above the shaded segments 506 and include “eyeball” icons in the shaded space that defines the markers. Although not always required, each observation marker preferably includes a text label that describes its meaning, relevance, or context (the labels shown in FIG. 5 are merely exemplary in nature, and they are not intended to be exhaustive or limiting in any way). Moreover, each observation marker has a leg or pedestal that extends to the horizontal timeline, which provides a timestamp for the observation. The temporal alignment of the observation markers and the various segments in the patient timeline 500 make it easy for a coach or an HCP to obtain a quick overview of the patient's status, behavior, and glycemic condition for the displayed period of time.

Notably, each observation marker represents an insight, and selection of an observation marker expands it to display additional detail related to the corresponding insight. In theory, the patient's coach will be able to understand the ongoing status of the patient with just a glance of the patient timeline 500 without expanding all of the observation markers. The text labels of the observation markers provide a good summary of what the patient is experiencing. Moreover, the observation markers can be graphically coded to convey additional information if so desired. For example, the observation markers can be color coded, shaded, animated, displayed with variable transparency, displayed with variable text fonts, or the like.

FIG. 5 is provided here to illustrate the format and arrangement of one appropriate patient report. It should be appreciated that the patient timeline 500 represents only one form of output that can be generated by the therapy management and coaching system described here. A variety of additional or alternative reports, created in different formats and layouts, can be generated and presented to the coach's device and/or to the trainee patient's device if so desired. As mentioned above, the system 102 can provide a website for navigation and interaction by the coach, wherein the coach can initiate the rendering of different reports, output formats, statistics, and the like.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A method of creating and managing interactive patient coaching programs, the method comprising the steps of:
collecting patient data associated with a plurality of medical device users, wherein the plurality of medical device users comprises a trainee patient assigned to a coach;
receiving a patient request for coaching;
processing the request with a computer-based system to automatically identify goals to be achieved by the trainee patient;
communicating the identified goals to a computer-based user device associated with the coach;
receiving, at the computer-based system, an accepted goal selected from the identified goals;
creating, with the computer-based system, a patient coaching program for the accepted goal;
generating insight messages based on patient data collected for the trainee patient, the generated insight messages related to the patient coaching program; and
delivering the generated insight messages to the computer-based user device associated with the coach.

2. The method of claim 1, further comprising the step of delivering at least some of the generated insight messages to a computer-based presentation device associated with the trainee patient.

3. The method of claim 1, wherein the step of processing the request comprises:
identifying a high level goal; and
identifying a number of low level goals that are subordinate to the identified high level goal.

4. The method of claim 1, wherein the step of processing the request comprises:
identifying at least one patient outcome goal for the trainee patient.

5. The method of claim 1, wherein the step of processing the request comprises:
identifying at least one patient behavior goal for the trainee patient.

6. The method of claim 1, further comprising the step of communicating the identified goals to a computer-based presentation device associated with the trainee patient.

7. The method of claim 1, wherein the insight messages include goal tracking insight messages intended for the coach and the trainee patient, observational insight messages intended for the trainee patient, and educational insight messages intended for delivery to the trainee patient at the discretion of the coach.

8. The method of claim 7, wherein:
the delivering step delivers goal tracking insight messages in accordance with a schedule that is specific to the patient coaching program;
the delivering step delivers observational insight messages on an as-needed basis; and
the delivering step delivers educational insight messages based on a review of delivered goal tracking insight messages and delivered observational insight messages.

9. The method of claim 1, further comprising the step of:
acquiring patient feedback data related to the patient coaching program.

10. The method of claim 1, further comprising the step of:
acquiring patient feedback data related to delivered insight messages.

11. The method of claim 1, further comprising the steps of:
preparing, with the computer-based system, a final report upon completion of the patient coaching program; and
communicating the final report to the computer-based user device associated with the coach and to a computer-based presentation device associated with the trainee patient.

12. The method of claim 1, wherein:
the medical device users are insulin infusion device users; and
the patient data comprises at least some of: carbohydrate amount; bolus information; insulin to carbohydrate ratio; insulin sensitivity factor; active insulin amount; time of day; basal rate; temporary basal use; consecutive boluses; insulin suspension; reservoir rewind time; reservoir priming time; pump alarms and associated alarm times; sensor alerts and associated alert times; blood glucose readings and associated measurement times; user demographic information; meal times and corresponding meal content; exercise times and corresponding exercise content or type; medication type, dosage, and time; sleep time and quality; stress time; and electronic medical records; medical lab test data.

13. A computer-implemented patient therapy management and coaching system comprising:
at least one processor device; and
a non-transitory processor-readable medium operatively associated with the at least one processor device, the processor-readable medium comprising executable instructions configurable to cause the at least one processor device to perform a method comprising:
collecting patient data associated with a plurality of medical device users, wherein the plurality of medical device users comprises a trainee patient assigned to a coach;
receiving a patient request for coaching;
processing the request to automatically identify goals to be achieved by the trainee patient;
communicating the identified goals to a computer-based user device associated with the coach;
receiving an accepted goal selected from the identified goals;
creating a patient coaching program for the accepted goal;
generating insight messages based on patient data collected for the trainee patient, the generated insight messages related to the patient coaching program; and
delivering the generated insight messages to the computer-based user device associated with the coach.

14. The system of claim 13, wherein processing the request comprises:
identifying a high level goal; and
identifying a number of low level goals that are subordinate to the identified high level goal.

15. The system of claim 13, wherein processing the request comprises:
identifying at least one patient outcome goal for the trainee patient; and
identifying at least one patient behavior goal for the trainee patient.

16. The system of claim 13, wherein the insight messages include goal tracking insight messages intended for the coach and the trainee patient, observational insight messages intended for the trainee patient, and educational insight messages intended for delivery to the trainee patient at the discretion of the coach.

17. A system comprising:
a computer-implemented patient therapy management and coaching system;
a database system, associated with the patient therapy management and coaching system, to collect and maintain patient data associated with a plurality of medical device users, wherein the plurality of medical device users comprises a trainee patient assigned to a coach; and
a computer-implemented user device communicatively coupled to the patient therapy management and coaching system, the user device associated with the coach;
the patient therapy management and coaching system operative to:
receive a patient request for coaching;
process the request to automatically identify goals to be achieved by the trainee patient;
communicate the identified goals to the user device associated with the coach;
receive an accepted goal selected from the identified goals;
create a patient coaching program for the accepted goal;
generate insight messages based on patient data collected for the trainee patient, the generated insight messages related to the patient coaching program; and
deliver the generated insight messages to the user device associated with the coach.

18. The system of claim 17, wherein processing the request comprises:
identifying a high level goal; and
identifying a number of low level goals that are subordinate to the identified high level goal.

19. The system of claim 17, wherein the insight messages include goal tracking insight messages intended for the coach and the trainee patient, observational insight messages intended for the trainee patient, and educational insight messages intended for delivery to the trainee patient at the discretion of the coach.

20. The system of claim 17, wherein:
the patient therapy management and coaching system provides a therapy management and coaching website to the user device associated with the coach; and
the therapy management and coaching website comprises at least one webpage that includes information related to the coaching program and the trainee patient.
